(19) United States
(12) Patent Application Publication
(10) Pub. No.: US 2021/0353757 A1
(43) Pub. Date: Nov. 18, 2021

(54) NANOBODY PREPARATION SYSTEM FOR ADMINISTRATION THROUGH INTERNAL AND EXTERNAL EPIDERMIS OF HEAD AND NECK AND PREPARATION METHOD AND USE

(71) Applicant: Nanolattix Biotechnology Co. Ltd., Taiyuan, Shanxi (CN)

(72) Inventors: Shaoping Li, Taiyuan, Shanxi (CN); Zhican QU, Taiyuan, Shanxi (CN)

(21) Appl. No.: 16/068,669

(22) PCT Filed: Oct. 26, 2017
(86) PCT No.: PCT/CN2017/107800

§ 371 Date: Apr. 9, 2021

(30) Foreign Application Priority Data
Oct. 28, 2016 (CN) 201610961773.5

(51) Int. Cl. A61K 47/32; A61K 47/42; A61K 47/38; A61K 47/34; A61K 47/24; A61K 47/26; C07K 16/28; C07K 16/32; C07K 16/22

(52) U.S. Cl. CPC A61K 47/32; A61K 47/42; A61K 47/38; A61K 47/34; B82Y 5/00; A61K 47/26; C07K 16/2863; C07K 16/32; C07K 16/22; A61K 47/24

(57) ABSTRACT

Disclosed is a nanobody preparation system for administration through the internal and external epidermis of head and neck and internal epidermal layer of nasal cavity, comprising a specific nanobody having a biological activity and an antibody-drug carrier for improving the stability and tissue penetration of the nanobody. The drug carrier comprises one or more of a water-soluble macromolecular bio-saccharide gum substrate, polyvinyl alcohol, polyamino acid, glycerine, phospholipid, sodium carboxymethyl cellulose, collagen, and a hydrolase inhibitor.

### TECHNICAL FIELD

The invention belongs to the field of medical technology and it is included of preparation system and the method of specific nanobody delivery system via the head and neck epidermis layer and the intranasal epidermis layer, a preparation method thereof, and application of the product is for the disease of central nerve system and related health care.

### BACKGROUND TECHNIQUE

The blood-brain barrier (BBB) is a barrier between plasma and brain cells formed by the cerebral capillary wall and glial cells, and a barrier between plasma and cerebrospinal fluid formed by the choroid plexus. The barrier can prevent certain substances from entering the brain tissue from the blood. It is a major obstacle to the treatment of brain diseases because most of drugs are blocked.

To utilize the therapeutic biological and chemical drugs on brain diseases, it is necessary to reach an effective concentration in the damaged or diseased brain. Due to the presence of the blood-brain barrier (BBB), most drugs cannot achieve effective therapeutic concentrations in brain tissues, especially administered orally.

The brain is essentially an independent ecosystem that has specialized anti-infection defense systems and unique waste removal procedures. The BBB tightly controls macromolecules entering and leaving the brain, allows the brain to handle biological waste while ingesting nutrients and oxygen.

The BBB consists actually endothelial cells. The multi-layered membrane formed by these endothelial cells tightly encloses all blood vessels in the brain and prevents the brain from contacting the toxic substances in the blood. The BBB can block bacteria, viruses, and all other toxic substances from entering the brain. While blocking the entry of harmful substances, the BBB can allow important molecules, such as glucose, to pass through specialized transportation mechanisms.

One of the disadvantages of the BBB is that it is completely shielded against most drugs, and only certain types of lipid and lipid-soluble compounds are free passing the BBB. Even though these drugs will enter the brain, it usually causes serious side effects.

On the other hand, current treatment of brain diseases includes surgery, radiation, chemotherapy, and local intra-tumor therapy. These methods of treatment all have certain defect and hidden dangers for the patients. It is essential to have a more reliable, easier, and effective method to treat brain diseases.

For example, it is difficult to treatment of neurodegenerative diseases because of BBB. At present, almost 100% of macromolecular drugs and about 98% of small molecule drugs are difficult to penetrate into the central nervous system (CNS) through traditional clinical drug administrations. It is not only difficult to penetrate the BBB by injection or oral administration, but may also cause side effects such as gastrointestinal reactions, arrhythmia, etc. If most of drug were distributed in other tissues inelderly patients, especially those suffering from AD, it may need to increase the time of administration.

The BBB is a difficult barrier for neuroscience to overcome. Finding effective, and particularly safe and reversible, ways to open the BBB is one of the main goals of neurological therapy development over the years. As you known, diseases such as brain cancers (malignant gliomas) and other cancers located in or peripheral of the brain are very difficult to treat and therefore mortality rate is very high.

The most direct way to treat a brain tumor is to remove it surgically. Surgery is effective in obtaining tissue for diagnosis and removing tumors from adjacent normal brains. However, it is invasive, expensive, and poses a risk to the patient of potential surgical complications. More importantly, surgery cannot treat advanced malignant brain tumors because cancer cells usually invade the normal brain at the time of diagnosis. In addition, surgery is only available when the tumor is in a surgically accessible location. Tumors located deep inside the brain are generally not suitable for surgery because surgery can severely impair the patient's neurological function. Even if surgery is practicable, there is still the risk of brain damage and the extremely long recovery time after surgery.

Radiation therapy is also an optional treatment for brain cancer. It is usually set to fractionated dose treatment, covering a certain area around the tumor over a period of several weeks. The spatial localized forms of radiation including the Cyber-knife and gamma knife have all been used and have achieved different levels of success. Although radiation is still widely recognized as an effective method for additional treatment of malignant brain tumors, it has the disadvantage that it has only a small fraction of application, because some radiated brain parts will lead severe consequences.

Systemic chemotherapy can be used to combat brain tumors. Systemic chemotherapy is a viable option as an adjunct to radiation and surgery. However, due to the BBB and the systemic side effects from chemotherapeutic agents, systemic chemotherapy is greatly limited in brain cancer.

In 1993, Belgian scientists firstly reported that about half of the antibodies in camel's blood had no light chain, and these heavy-chain antibodies (HCAbs) lacking the light chain can be closely bound to antigens and other targets alike normal antibodies. In addition, it is unlike scFv sticking to each other and form clusters.

The camel single chain antibody only contains one variable domain of heavy chain of HCAb (VHH) and two conventional constant regions CH2 and CH3 regions, and more importantly, the VHH region cloned and expressed separately. With good stabile structure and antigen binding activity, VHH is the smallest unit known to bind targeting antigens, so VHH is also known as Nanobodies. Camel single chain antibodies are characterized by high affinity and high specificity, while immunogenicity (although non-human, but very low immunogenicity) and toxicity is very low, it is not easy to stick together and form cluster.

Compared with the heavy chain variable region VH of the human antibody, the CDR3 of the cluster complementary region of the nano-antibodies is longer, can form a convex ring structure, and can penetrate the antigen better inside the antigen, and thus has a higher affinity. In addition, hydrophobic residues of nanobodies are replaced by hydrophilic residues, which are more water soluble and less likely to form clusters.

Nanobodies are currently the smallest unit known that can bind a target antigen. VHH crystal size 2.5 nm×4 nm, molecular weight is only 12 KD-15 KD, its molecular structure is relatively stable, able to withstand high temperatures and maintain activity in extremely harsh environments. Studies have confirmed that VHH can retain 80% of its biological activity after being left at 37° C. for 1 week, indicating that the Nanobodies is very stable at room temperature, making it easier to store and transport than that of conventional antibodies.

Nanobodies have a strong and rapid skin tissue penetration ability, and can effectively penetrate the brain via neck skin and nasal mucosa, which provides a new method for brain tumors

Also, the nanobody has reversible refolding ability, ie, recovery. The experiment shows that the nanobody still maintains a high activity after being treated at a high temperature of 90° C., and can regain the ability to bind target antigen. All conventional antibodies lose their activity after 90° C. treatment and irreversible polymerization occurs. Normal antibodies can fail or break down under severe conditions, such as chaotropic agents, presence of proteases, and extreme pH denaturation, while nanobodies still have a high degree of stability.

In addition, nanobodies also exhibit a characteristic of being less prone to denaturation or variability after denaturation under the conditions of a strong denaturant.

Compared with conventional antibodies, 1), Nanobodies are easily obtained and readily achievable, which can be obtained by means of immunization, B lymphocyte isolation, antibody library display technology screening, etc.; 2), good stability, its internal fold contains more than two sulfur bond, which makes its structure have good stability, can be sored at room temperature; 3), high solubility, unlike scFv as easy to aggregate into clusters, nanobody is hydrophilic, has good water solubility, can be improved as drug utilization; 4) absorption is good, due to high solubility, nanobodies have the advantage of high absorption rate; 5) Nanobodies are easy to express, unlike traditional antibodies, which must be expressed in mammalian cells with low yield and high cost, VHH can be highly expressed in prokaryotic cells, some researchers have increased the output to 2.5 g/L; 6), simple humanization, homology with human heavy chain genes in 80-90%, Humanization has been successful; 7) Nanobodies easily cross biofilm systems and are also easy to couple with other molecules.

With development progress of bioengineering technology, a large number of nanobody drugs will continue to emerge. At present, the main administration ways for clinical trials are injections and oral administration. Due to the small molecular weight of the nanobodies, it has poor stability in vivo and is susceptible to degradation by the gastrointestinal tract and the first-pass effect of the liver enzyme system during oral administration. To achieve an effective drug therapeutic concentration, the patient needs long-term and multiple repeated injections or oral administration.

Transdermal head and neck epidermal delivery system refers to a controlled release drug delivery system that can enhance the effectiveness of the drug by penetrating the head and neck cortical tissue, or the nasal mucosa, into the intracranial system. It can avoid gastrointestinal absorption difficulties caused by the interaction of gastrointestinal pH, enzymes, food and other drugs, and avoid first-pass effects. It can also avoid the inconvenience and inefficiency caused by injection administration. It prolongs the efficacy after a single administration, and extends the treatment time of short-lived drugs through drug storage and controlled release characteristics.

Due to the special physical and biological characteristics, nanobodies can effectively penetrate the head and neck epithelial tissue and nasal mucosal endothelial tissue into the brain to achieve transdermal intracranial administration. Nanobody is a safe and effective method of administration through inner and outside the epidermis. It is an innovation in methods and administration forms of biopharmaceuticals.

The BBB is the last obstacle that neuroscience will be overcome via utilizing nanobodies to pass the BBB and to treat brain tumors. Also Alzheimer's disease could be treated in the same fashion. This method will be a great advancement in the treatment of malignant encephalopathy such as cancer, Parkinson's as well as Alzheimer's disease.

The human nasal cavity has a unique relationship with the cranial cavity in terms of anatomy and physiology. The olfactory nerve epithelium is the only tissue in which the CNS is in direct contact with the outside of brain. An axon-forming bundle of olfactory sensory neurons covered by nasal cilia can pass through the sieve plate into the cranial cavity and form a synaptic connection with the mitral and tufted cells of the olfactory bulb in the brain. The human brain absorbs drug from the nasal cavity via olfactory mucosal epithelia. After nasal administration, the drugs stay in the olfactory mucous membrane and are easily absorbed into the human cerebrospinal fluid. Therefore, the drug can bypass the BBB to enter into the CNS for a therapeutic role.

Due to the connect of the olfactory nerve and the olfactory mucosa epithelia described above, the nasal cavity has become an effective route for the non-invasive delivery of drugs to the brain. However, due to poor transmembrane ability of some drugs, direct administration in the form of a solution is susceptible to be degraded by the enzymes in the nasal cavity and removed by nasal cilia. Therefore, the amount of the drug that enters the brain through the nose is still low, and an effective clinical treatment effect cannot be achieved. Therefore, the long-term contact time of the nanobody drug with the nasal mucosa delays the elimination of the drug, and can increase the effective intracerebral concentration of the drug for intranasal administration.

The content of proteolytic enzymes in nasal mucosa tissue is little, which is conducive to maintaining the stability of nanobody drugs. Nanobody drugs have small molecular weight, small size, and stable structure and biological activity. They can easily penetrate the brain and nasal mucosa and cortex tissue of the brain to achieve an ideal transdermal absorption effect, enter the blood circulation system of the brain, and reach the target area of disease.

The preparation and delivery system of the nanobody via the head and neck outer epidermis layer and the nasal mucosa inner surface has the advantages of high curative effect, convenient administration, non-invasiveness and painlessness, and the product preparation process is simple and suitable for industrialized production. There are no relevant reports in China and abroad.

### SUMMARY OF THE INVENTION

The purpose of the present invention is to provide a system for administering a nanobodies through the head and neck outer epidermis and the nasal epidermis, which is non-toxic, low cost, and convenient to use, and is particularly suitable for nanobodies. The drugs are mixed with other drugs through the outer epidermis of the head and neck and the inner epidermis of the nasal mucosa.

In addition, the purpose of the present invention is also to provide a method for preparation and application of the above head and neck inner and outer epidermis delivery systems.

The nanobodies of the present invention comprises a biologically active specific nanobodies in a system for transdermal administration of drug from nasal mucous membranes to the brain, and an antibody drug carrier for increasing stability and tissue penetration of the nanobodies. Antibody drug carriers include, but are not limited to, water-soluble macromolecule biosaccharide gum bases, polyvinyl alcohol, polyamino acids, glycerol, phospholipids, gelatin, sodium carboxymethyl cellulose, collagens, and hydrolase inhibitors. Or a mixture of several proportions, the specific nanobodies includes humanized and non-humanized antibody forms. The nanometer antibody is administered to the epidermis and nasal mucosa for getting into the brain differently from the injection dosage form and the oral dosage form, and is an effective drug component that is administered through the epidermis and nasal mucous membranes of the brain, has no pain, self-administration, and can be used any time treatment. Compared with the injection dosage form, it is simple to use and is high efficiency.

In the present invention, the antibody drug carrier is composed of a water-soluble polymer bio-glycosyl matrix, polyvinyl alcohol and other matrix materials mixed with water, such antibody drug carriers carrying a large amount of drugs, and a variety of drugs including plant extracts and chemical drugs with good affinity. After sustained release of the drug via epidermis and nasal mucosa to the brain, the epidermis has good transdermal effects, breathability, and no-irritation was found for application. There is basically no allergic reaction. It is an ideal classic. Head and neck epidermis and nasal mucous membrane delivery platform, combined with a variety of drugs, can provide a variety of sustained drug delivery system.

Furthermore, preferred antibody drug carriers of the present invention are collagens, including collagen peptides, collagen, and gelatin. The combination of collagen as a nanobodies application can be used to treat other brain diseases via the epidermis and nasal mucosal drug delivery systems.

Collagen is a polymer functional protein. It is the main component of the skin. It accounts for 80% of the dermal layer of the skin. It forms a fine elastic network in the skin. It firmly locks in moisture and supports the skin. Collagen is composed of three peptide chains and spiral fibrous protein. It is also the most abundant protein in the human body. It is widely distributed in the connective tissue, skin, bone, visceral interstitial and muscle cavity, ligament, sclera and other parts of the body. 30% or more of the proteins in human body needs proline, hydroxyproline and other collagen characteristic amino acid and collagen is an important part of human cells, especially skin extracellular matrix.

Collagen can be subdivided into: macromolecule collagen and small-molecule collagen peptide. The trotter gum we eat usually contains collagen but it is a large molecule protein with a molecular weight of 300,000 Daltons. It cannot be directly absorbed by the body and its absorption rate is very low; and collagens whose molecular weight is controlled within 6,000 Daltons by techniques such as acid-base and enzyme digestion are called collagen peptide peptides which are intermediate amino acids and macromolecules. Substances between proteins, two or more amino acids dehydration condensation to form a number of peptide bonds to form a peptide, multiple peptides are multi-folded to form a protein molecule. Peptides are precise protein fragments, the size of which is only nanometer-sized. Blood vessels and skin are easily absorbed, and their absorption rate is much higher than that of macromolecular proteins.

The skin has a good absorption effect on collagen peptides. The molecular weight of collagen peptide peptides is small, and the polypeptide chains of different peptide segments do not have hinges with each other but have a linear structure. Experiments show that the absorption rate of collagen peptide peptides is between 95% and 100%, while the absorption rate of collagen is between 0% and 2.5%. Collagen peptides have good solubility. Collagen peptides contain a large number of hydrophilic groups (—COOH, —NH2, —OH), which enhances the interaction between polypeptides and water molecules, and their water solubility is greatly increased. Collagen peptides are safe and have no side effects. Collagen peptide itself is a protein without any side effects.

In the present invention, the optimized antibody drug carrier is composed of the following parts by weight: 8 parts of water-soluble macromolecular bio glycogum matrix, 8 parts of collagen, 10 parts of collagen peptide, 10 parts of polyhydroxy compound, 5 parts of polyvinyl alcohol, 10 parts of levoacetic anhydride, 10 parts of poly amino acid, 10 parts of glycerol, 5 parts of phospholipid, 2.5 parts of gelatin, 1 part of sodium carboxymethyl cellulose and 20 parts of water.

The method for preparing the drug delivery system of the present invention by using the antibody drug carrier is to add the weight parts of polyvinyl alcohol into the weight parts of water, heat and stir at 95° C. for 45 minutes to completely dissolve the polyvinyl alcohol, and then add the water-soluble macromolecular biological glycogum matrix, collagen, collagen peptide, polyhydroxy compound, levoacetic anhydride and polyaminoacid in turn After heating to 60-70° C. for 15 min, glycerin, phospholipid, gelatin and sodium carboxymethylcellulose were heated to 60-70° C. and stirred for 15 min to completely dissolve and mix evenly. When the temperature dropped to 10-20° C., the specific nano antibody dosage needed was added to prepare the nano antibody delivery system through the inner and outer epidermis of head and neck.

In the present invention, the specific nanobodies include an active nanobody, a nanobody fragment, or a multiple target nanobody polymerized linker.

The nanobodies of the present invention may also be an expressed and purified nanobody, or an active nanobody expressed and expressed by an active probiotic microorganism in a drug delivery system, a multiple targeted nanobody polymerized linker, and a nanobody and a protein couple. Conjugates of nanobodies and drugs are expressed and secreted adjacent to the head and neck epidermis as well as nasal mucosa.

Specifically, in the above-mentioned nanobodies polymerized linkers, the same or different nanobodies may be polymerized, or the nanobodies may be combined with albumin to prolong the half life in the antibody drug carrier and human body. The half-life of nanobodies is not very long and measures need to be taken to prolong their half-life in antibody drug carriers and brain. The nanobody preparation administered through nasal cavity mucosa to the brain of the present invention is modified by the fusion of nanobody and albumin, Fc fusion, pegylation (PEG), etc. The nanobody is present in the antibody drug carrier and on the nasal mucosa, the half-life can be greatly extended.

The nanobodies according to the present invention is applied to a system for administration to the upper and nasal mucosa of the brain, and the nanobodies is a specific nanobody that targets different lesions. The target of the lesion includes, but is not limited to, various types of brain tumor, malignant glioma, meningitis, cerebrovascular disease, migraine. Cerebral skin disease, Alzheimer's disease, atopic dermatitis, nasal cancer, chronic sinusitis, etc.

Specifically, the nanobodies-specific disorder target includes, but is not limited to, HER2 (human epidermal growth factor receptor 2 or HER2/neu), EGFR (epidermal growth factor receptor), VEGF (vascular endothelial growth factor), VEGFR, FGFa (epidermal growth factor a), FGFb (epidermal growth factor b), TNFa (Tumor Necrosis Factor a), TNFb (Tumor Necrosis Factor b), PD-1, PD-L1, CTLA4, Sclerostin, Glucagon-like peptide 1, and Glucagon-like peptide receptor, interferon IL-4, IL-5, IL-6, IL-9, IL-13, IL-17a, etc.

Furthermore, the nanobodies of the present invention may include one or more specific nanobodies in the system for head and neck epidermis and nasal cavity of the brain, and may be combined with other kinds of drugs to form a complex drugs delivery system.

The present invention prepares the nanobodies via head and neck epidermis and nasal mucous delivery system for the brain by means of creams, hydrogels, gels, nasal effervescent granules, nasal powders, nasal creams or nasal lotions, etc. A variety of topical dosage forms suitable for administration to and from the nasal mucous into the brain. Nanobodies are dispersed in water, gel or cream and applied to the skin of the brain to release active nano-particles in the head and neck epidermis and nasal mucosa. The antibody or antibody fragment enables active nanobodies or antibody fragments to efficiently penetrate the head and neck epidermis and nasal mucosa.

Specific applications of the nanobodies of the present invention via head and neck epidermis and nasal mucosal drug delivery systems include the action of the agent being absorbed through nasal mucosa to the brain, into the blood circulation of the brain to reach the lesion site.

More explaining in detail, it includes the application of the preparation that does not need to cross the BBB, and the nanobody enters the intracranial brain tissue and central nervous system through the outer cortex of the head and neck and the inner epidermis of the nasal cavity to reach the preset lesion target and play the role of drugs. It includes the application of the preparation which can make nanobody penetrate the inner and outer epidermis of the head and neck, penetrate the BBB, enter the blood circulation, and reach the preset central nervous system lesion site.

The nanobody of the present invention can be used to treat autoimmune diseases through brain and nasal mucosal drug delivery systems, including Alzheimer's and cancer.

The amyloid and Tau proteins were found in the brain of Alzheimer's patients. The abnormal accumulation of these two proteins can affect the normal function of neurons and lead to the apoptosis of nerve cells. Therefore, the use of nanobodies activate the body's own immune system, and clear these two proteins out of the brain.

After entering the brain via head and neck epidermis and the nasal mucosa, the nano antibody specific to amyloid beta protein enters the brain, it can selectively bind to amyloid plaques in the brain, and then activate the immune system to clean up the deposited proteins from the brain.

In general, certain expression of lesions at the site of a neurodegenerative disease and/or certain receptors of the BBB may be altered by the nanobodies. Nanobodies may be used for targeting lesion such as low-density lipoprotein which are body-associated proteins were highly expressed at the lesion site and BBB. The use of the above-mentioned protein ligand nanobodies as a targeting molecule can help carrying the drug to cross the BBB, and concentrate in the lesion areas of AD and PD, thereby improving the therapeutic effect and reducing the toxic and side effects.

The nanobodies of the present invention may also be used as a special health care product for the elderly head and neck epidermis and nasal mucosal delivery systems. For example, the nanobodies may be used to apply nutrition to the brain through the skin and nasal mucosal drug delivery systems.

The prevention and treatment of neurodegenerative diseases are faced with many difficulties. In traditional drug therapy, the treatment of neurodegenerative diseases is difficult due to the BBB. At present, almost 100% of macromolecular drugs and about 98% of small molecule drugs are difficult to enter the CNS through the BBB. Systemic administration via injection or oral administration is not only difficult to penetrate the BBB, and may also cause side effects such as gastrointestinal reactions, arrhythmia, etc. Due to a large amount of distributions in other tissues, and elderly patients, especially those suffering from AD, are compliant with medications.

The nanobody drug through the BBB into the CNS to achieve the way include: 1 as a targeted drug, via head and neck epidermis and nasal mucosa, bypass the BBB into the CNS, to achieve cross-cell transport; 2 directly penetrate the brain between the epidermis and cell diffusion; 3 directly penetrates the blood vessels of brain lesions to spread across cells; 4 albumin and histones cross-cell transport through adsorption. BBB is a structure in the brain capillaries that prevents certain substances from entering the brain tissue from the blood and is a close structure composed of brain capillary endothelial cells, the terminal pod of astrocyte, pericytes, and vascular basement membrane. It can selectively absorb essential substances for the brain, discharge harmful or excess substances at the same time, maintain the stability of the brain environment, protect the brain tissue, and maintain the normal physiological functions of the CNS.

### Invention Beneficial Effect

The present invention discloses, for the first time, a delivery system that uses specific nanobodies via head and neck epidermis and nasal mucosa and its preparation method and application. Nanobodies are the smallest unit known to bind target antigens, and their three-dimensional structure determines the biological properties such as biological stability and relative stability of nanobodies. The invention optimizes the carrier formula, maintains the stability and the tissue penetrability of the nanobodies, and realizes the wide application of the nanobodies through the head and neck epidermis and the nasal mucosal delivery system for clinical treatment and the elderly health care field. The nanometer antibody of the present invention is a safe and effective administration method via head and neck epidermis and nasal mucosal drug delivery system, and opens up new drug administration forms of nanobodies biopharmaceuticals, and its application prospect is very broad.

### Invention Implementation Examples

The following examples illustrate specific embodiments of the present invention. However, it should be noted that the following are only examples or illustrations for the application of the present invention. Those skilled in the art can devise many variations and alternative compositions, methods, and systems without departing from the spirit and scope of the present invention. The appended claims are intended to cover these changes and arrangements. Therefore, although the present invention has been described in detail below, it only provides one of the details of an embodiment of the present invention. Without departing from the core content of the present invention: the concept that nanobodies are conceived via head and neck epidermis and nasal mucosal delivery system, several modifications and improvements made should fall within the protection scope of the present invention.

### EXAMPLE 1

The purpose of this embodiment is to provide a sustained-release administration system for head and neck epidermis and nasal mucosa using a water-soluble polymer protein material as a main substrate, a preparation method of the drug delivery system, and the administration system application.

The drug delivery system is a head and neck epidermis and nasal mucosal delivery system using a water-soluble polymeric biosaccharide gum as the main antibody drug carrier. In this delivery system, the weight of a typical antibody drug carrier were: 8 parts of a water-soluble macromolecule bioglycogen matrix, 8 parts of collagen, 10 parts of collagen peptide, 10 parts of polyhydroxy compounds, 5 parts of polyvinyl alcohol, 10 parts of dextran, 10 parts of polyamino acids, 10 parts of glycerol, 5 parts of phospholipids, 2.5 parts of gelatin, 1 part of sodium carboxymethyl cellulose, and 20 parts of water.

For different nanobodies, the above antibody drug carriers can be adjusted accordingly to better maintain and increase the stability and tissue penetration of the nanobodies.

In the present embodiment, the above-mentioned sustained-release administration system for the skin and nasal cavity of the brain using a water-soluble macromolecular biosaccharide gum as a main antibody drug carrier can be prepared by the following method: according to the prescription ratio, the water solubility is measured. The macromolecule biosaccharide matrix and the polyvinyl alcohol are added to an appropriate amount of water, and the mixture is heated and stirred at 95° C. for 45 minutes to completely dissolve the polyvinyl alcohol, and the water-soluble macromolecule biosaccharide matrix, the collagen, and the collagen are added in an order. Peptides, polyhydroxy compounds, dextran, polyamino acids, glycerol, phospholipids, gelatin and sodium carboxymethyl cellulose, heated to 60-70° C., stirred for 15 minutes, completely dissolved and mixed until the temperature dropped to 10-20° C., adding specific nanobodies of the required concentration to prepare an in vitro drug delivery system.

The above-mentioned transdermal system for epidermis and nasal mucosa can also be further processed into creams, cross-linked PEG hydrogels, gels and other dosage forms.

The above-mentioned transdermal system for the epidermis and nasal mucous membranes of the brain is also processed into a foaming nasal cavity granules, a nasal powder, a nasal cream, and a nasal lotion.

### EXAMPLE 2

In the present embodiment, collagen is used as an antibody carrier to prepare methods related to nanobodies via head and neck epidermis and nasal mucosal delivery systems.

The collagen may be a collagen peptide, collagen, or gelatin.

The collagen peptide used in this embodiment can be prepared by enzymatic hydrolysis of gelatin, ejiao, new gelatin, pork skin, bovine, pig bone, bovine bone, pig Achilles, or bovine Achilles' tendon. The relative molecular weight is not higher than 100 kD.

The collagen used in this example can be obtained by enzymatic hydrolysis of pig skin, bovine, pig bone, bovine bone, pig Achilles' tendon or bovine Achilles' tendon, and its relative molecular weight is 250-300 kD.

In the system for administering the nanobodies prepared in this embodiment via head and neck epidermis and nasal mucosa of the brain, the ratio by weight of the active ingredient of the nanobodies to the collagen is 0.001 to 0.1:1, and the preferred weight ratio is 0.1:5 to 50. Here, the weight ratio of active ingredient of nanobodies to collagen refers to the ratio of the weight of both solids in the final product.

In this embodiment, the collagen used by the nanobodies collagen via head and neck epidermis and nasal mucosal drug delivery system may be collagen peptide or collagen alone, and may also be a mixture of collagen peptide and collagen. The two properties are similar, but there is a certain difference in molecular weight. Collagen is a helical fibrous protein twisted from three peptide chains and is an important protein that constitutes the connective tissue of animals. It is mainly hydrolyzed from raw materials such as pig skin, cowhide, bovine Achilles' tendon, pig Achilles' tendon, pork bone, and bovine bone.

The collagen used in the present embodiment is generally digested with a single enzyme such as pepsin, papain, and is usually performed at a low temperature (below 1° C.) to prevent protein denaturation without destroying the unique right-handed helix. The main body of the structure only excised the non-collagenous caudal peptide, which has a relative molecular weight of 250-300 kD and a complete triple helix structure. The collagen is further hydrolyzed to obtain gelatin, which has a relative molecular weight of 100-200 kD and the triple helix structure has been destroyed. Collagen peptide is a further enzymolysis product of gelatin, its relative molecular weight is below 100 kD, and most of it exists in the form of collagen oligopeptides with relative molecular weight below 40 kD.

The collagen used in this embodiment can also be directly obtained from the enzymatic hydrolysis of gelatin. Collagen and gelatin have relatively high molecular weight, poor water solubility, high viscosity, high mechanical strength, good auxiliary drug formability, ability to adhere to wounds, and can be enzymatically digested into oligopeptides or amino acids involved in tissue growth under the action of collagenase; The relatively small molecular weight of collagen, contains more collagen oligopeptides, water-soluble, easy to be absorbed and used by the body, and due to more proline and hydroxyproline content can be directly involved in the body's tissue cells grow, provide it with raw materials. Collagen, gelatin and collagen peptides can all play a role in the promotion of wound healing and tissue repair in external preparations of traditional Chinese medicine.

The main raw material of the collagen peptide used in this embodiment is derived from any one or more of gelatin, new donkey hide, pig skin, cow leather, pork bone, bovine bone, pig Achilles' tendon, or cow heel. By enzymatic hydrolysis, the enzymatic hydrolysis method may be single enzymatic hydrolysis of pepsin, trypsin, trypsin, neutral protease, alkaline protease, and papain. The enzymatic hydrolysis of pepsin, trypsin, trypsin, neutral protease, alkaline protease, and papain can also be a biomimetic enzymatic method that completely imitates the human body's digestion and absorption process (Firstly, pepsin enzymatic hydrolysis method is used to simulate the digestive process and physicochemical parameters of the human stomach, and then the digestive absorption process and physicochemical parameters of pancreatic (protein) enzyme digestion are performed to obtain low molecular oligopeptides that can be directly absorbed by the human body. substance).

The main preparation method of the collagen peptide used in this embodiment is: taking collagen raw materials, clean water or salt solution, and remove impurities such as fat, residual meat, add acid or alkali to adjust the appropriate pH, low temperature (0˜1° C.) enzymatic hydrolysis for 24 to 72 hours, centrifugation, taking supernatant, adjusting the appropriate pH value, adding the required protease for full enzymolysis, taking the supernatant, dialysis salting out with neutral salt, and remove impurity.

The main raw material of the collagen used in the present embodiment is derived from any one or more of pigskin, cowhide, pig bone, bovine bone, pig Achilles' tendon, or bovine Achilles' tendon, and is obtained through proper enzymatic hydrolysis method, and its relative molecular weight 250 to 300 kD. The enzymatic hydrolysis method may be single enzymolysized of pepsin, trypsin, neutral protease, alkaline protease, papain, or it may be pepsin or trypsin. Enzymatic hydrolysis maybe for the several enzymes includes trypsin, neutral protease, alkaline protease, and papain.

The main preparation method of the collagen used in the present embodiment is: taking collagen raw materials, clean water or salt solution, and remove impurities such as fat, residual meat, add acid or alkali to adjust the appropriate pH, low temperature (0˜1° C.) Enzymatic hydrolysis of 24 to 72 hours, centrifugation, collect the supernatant, plus neutral salt salting, dialysis impurities, further purification.

In the preparation process of the collagen peptide or collagen described in the present embodiment, the acid used is any one or more of malic acid, citric acid, acetic acid, or synthetic acid-base water with adjustable pH values. Alkali is a pH-adjustable synthetic acid-alkali water.

In the preparation process of collagen peptide or collagen described in this embodiment, the salt used for salting out may be any one or more of ammonium sulfate, magnesium sulfate, sodium sulfate, sodium chloride, or sodium phosphate.

In the preparation process of the collagen peptide or collagen described in the present embodiment, if the alkali used is an artificial acid-alkali water with an adjustable HP value, no salting out process is needed, and the preparation process will be non-toxic and non-polluting.

### EXAMPLE 3

The nanobodies are administered via head and neck epidermis and nasal mucous membranes. Nanobodies in the delivery system can be used to eliminate IL-1 alpha (Interleukin 1 alpha) TNF-alpha, IL-8 (Interleukin 8), etc. inflammatory factors.

### EXAMPLE 4

The nanobody is administered via head and neck epidermis nasal mucous for transdermal administration. Nanobodies in the delivery system are gelled to eliminate infectious bacteria such as Propionibacterium acnes (P. acnes).

### EXAMPLE 5

A drug delivery system for nanobodies via head and neck epidermis and nasal mucosa is based on an anti-tumor nanobody-targeted targets EGFR, HER2, VEGFR2, c-Met, CXCR7, etc., and can be applied to the head and neck epidermis and nasal mucosa. It can also form nanoparticles that penetrate in the cancerous area and is used to treat cancer.

### EXAMPLE 6

The nanobody is delivery system for transdermal administration via head and neck epidermis and nasal mucosa, which is a gel based on a trivalent nanobody that specifically inhibits TNFR1 and can be used for anti-inflammatory.

### EXAMPLE 7

The nanobodies specific for beta amyloid protein, enters the brain through nasal mucosa. In order to effectively treat Alzheimer's disease and dementia, it can selectively bind to the amyloid plaques in the brain, and then activate the immune system to clean the deposited protein out of the brain.

Nanobodies of the invention via head and neck epidermis and nasal mucosal delivery systems may also include its activation on microorganisms in the brain. Where the antibody or antibody fragment can be expressed and/or secreted on the surface of the head and neck epidermis and the mucosal surface of the nasal cavity.

Any of the above-described nanobodies through skin and nasal mucosal drug delivery systems of the present invention, wherein the antibody is a VHH-type or VNAR-type heavy chain immunoglobulin or a fragment thereof, preferably derived from Alpaca Camelids, most preferably derived from a llama heavy chain antibody or fragment thereof, or the antibody is a domain antibody (dAb) or fragment thereof of an immunoglobulin heavy chain or light chain.

The nanobodies via head and neck epidermis and nasal mucosal delivery system of the present invention can improve bioavailability, reduce dosage, reduce adverse reactions, increase drug treatment index, increase clinical drug safety and formulation compliance. Therefore, the drug delivery system of the present invention has advantages superior to those of conventional drug delivery systems. However, there is currently no application of the nanobodies via head and neck epidermis and nasal mucosa delivery system described in the present invention.

One of the nanobodies nasal mucosal gel formulations is a weakly cross-linked polymer that expands upon contact with water and expands on the mucosal surface. They can adhere tightly to the membrane at the site of absorption, put the drug delivery system at a certain location, and extend the residence time. Although nasal mucosal glands do not produce mucinous glandular cells, they are not strictly mucosal epithelium.

The mucosal adhesion has three steps: 1), affected by the surface energy effect and the expansion process; 2) the polymer chain through the polymer mucous membrane contact surface is unwound from the entangled state; 3) the exposed active site combined with tissue macromolecules. The dry hydrogel adheres to the moist tissue and is quite strong. It absorbs water from the tissue surface to dehydrate the surface and reduce the surface pressure. It adsorbs on the mucosal surface like an anchor, thus prolonging the local activation time of the drug.

The following lists some of the nanobodies VHH sequences that can be implemented in the present invention, which have been publicly reported. However, the nanobodies VHH sequences suitable for use in the present invention are not limited thereto.

The 128 amino acid VHH-αHER2 sequence 1:
DVQLVESGGG,
SVQGAAGGSL,
RLSCAASDIT,
YSTDCMGWFR,
QAPGKEREGV,
ATINNGRAIT,
YYADSVKGRF,
TISQDNAKNT,
VYLQMNSLRP,
KDTAIYYCAA,
RLRAGYCYPA,
DYSMDYWGKG,
TQVTVSSG.
The 126-amino acid VHH-αHER2 sequence 2:
DVQLEESGGG,
SVQTGGSLRL,
SACASGYTYS,
SACMGWFRQG,
PGKEREAVAD,
VNTGGRRTYY,
ADSVKGRFTI,
SQDNTKDMRY,
LQMNNLKPED,
TATYYCATGP,
RRRDYGLGPC,
DYNYWGQGTQ,
VTVSSG.
VHH-αVEGF sequence containing 132 amino acids
1:
MAQVQLQESG,
GGSVQDGGSL,
RLSCAASGYA,
YDTYYMGWFR,
QAPGKEREWV,
AGITSLVSGV,
AYYKYYTDSV,
KGRFTIFRDD,
DKNTVDLQMN,
SLKPEDTAIY,
YCAASRSGLR,
ARLLRPELYE,
YWGQGTQVTV,
SS.
VHH-alpha VEGF sequence 2 containing 129 amino
acids:
MAQVQLQESG,
GGSVQAGGSL,
RLSCVASGDT,
YSSACMGWFR,
QAPGKEREGV,
ATICTSTSMR,
TRYYADAVKA,
RFTISQDNAK,
NTVYLQMNSL,
KPEDIAMYYC,
ATGHTVGSSW,
RDPGAWRYWG,
QGTQVTVSS.
The 138-amino acid VHH-alpha EGFR sequence 1:
QVQLQESGGG,
LVQPGGSLRL,
SCASSGRTFS,
SYAMGWFRQA,
PGKQREFVAA,
IRWSGGYTYY,
TDSVKGRFTI,
SRDNAKTTVY,
LQMNSLKPED,
TAVYYCAATY,
LSSDYSRYAL,
PQRPLDYDYW,
GQGTQVTVSS,
LEHHHHHH.

The above non-humanized nanobodies VHH sequences can be humanized by modifying the amino acid sequence of the naturally occurring VHH sequence domain, ie one of the amino acid sequences of its naturally occurring VHH sequence domain. The above amino acid residues are replaced with amino acid residues existing at the corresponding positions in the conventional human VH sequence domain.

1. The nanobody drug delivery system via head and neck epidermis and nasal mucosal comprises specific nanobodies with biological activity and antibody drug carriers for maintaining the stability and tissue penetration of the nanobodies. The nanobody drug carriers include but are not limited to water-soluble macromolecular biological glycogum matrix, polyvinyl alcohol, poly amino acids, glycerin, phospholipid, sodium carboxymethyl cellulose, sodium carboxymethyl cellulose, sodium carboxymethyl cellulose, sodium carboxymethyl cellulose, sodium carboxymethyl cellulose, sodium carboxymethyl cellulose, sodium carboxymethyl cellulose, sodium carboxymethyl cellulose, sodium carboxymethyl, one or a mixture of collagen and hydrolase inhibitors in any proportion, and the specific nanobody includes humanized and non-human nanobody forms;

2. The drug delivery system according to claim 1, which is characterized in that the collagen includes collagen peptide, collagen and gelatin;

3. The drug delivery system according to claim 2, which is characterized in that the antibody drug carrier is composed of 8 parts by weight of water-soluble macromolecular bio glycogen matrix, 8 parts of collagen, 10 parts of collagen peptide, 10 parts of polyhydroxy compound, 5 parts of polyvinyl alcohol, 10 parts of levoglycan, 10 parts of Poly amino acid, 10 parts of glycerol, 5 parts of phospholipid, 2.5 parts of gelatin and carboxymethyl fiber Vitamin sodium 1 part, water 20 parts;

4. The preparation method of the drug delivery preparation system of claim 3 is that the weight parts of polyvinyl alcohol are added into the weight parts of water, heated and stirred at 95° C. for 45 minutes to completely dissolve the polyvinyl alcohol, and then the water-soluble high molecular biological glycogum matrix, collagen, collagen peptide, polyhydroxy compound, levoacetic anhydride, polyamino acid, glycerin and phosphorus are successively added Fat, gelatin and sodium carboxymethylcellulose were heated to 60-70° C. and stirred for 15 min to completely dissolve and mix evenly. When the temperature dropped to 10-20° C., the specific nano antibody was added to prepare the nano antibody delivery system via head and neck epidermis and nasal mucosa;

5. The drug delivery system of claim 1, wherein the specific nanobody comprises an active nanobody, a nanobody fragment and a genetically engineered nanobody, or a multiplexed nanobody polymer linker;

6. The drug delivery system according to claim 5, wherein the nanobody polymeric linker comprises a polymerization between the same or different Nanobodies, including the binding of the nanobody and the fusion protein to prolong the half-life of the drug in vivo including the combination of nanobody and chemical small molecule drug.

7. The drug delivery system according to claim 1, 2, 3, 5 or 6, which is characterized in that the specific nano antibodies in the delivery system are specific nano antibodies for different lesions, and the lesion targets include inflammation of head and neck skin, nasal cavity and ear cavity environment and adjacent organs, viral bacterial infection, head and neck tumors, brain neurodegenerative diseases, and alts Alzheimer's disease, Parkinson's disease, amyotrophic lateral sclerosis and Huntington's disease, various brain tumors and cancer, malignant glioma, meningitis, cerebrovascular disease, migraine, scalp disease, specific dermatitis, Alzheimer's disease, nasal tumor and cancer, chronic sinusitis;

8. The drug delivery system according to claim 7, which is characterized in that the disease targets targeted by the nano antibody include but are not limited to HER2, EGFR, VEGF, VEGFR, FGFa, FGFb, TNFa, TNFb, PD-1, PD-L1, CTLA4, sclerostin, glucagon like peptide 1, glucagon like peptide Receptor, interferon IL-4, IL-5, IL-6, IL-9, IL-13, IL-17A, and bacterial and viral infections;

9. The drug delivery system according to claim 1, 2, 3, 5 or 6, which is characterized in that the delivery system contains one or more specific nano antibodies and can be combined with other drugs to form a nano antibody delivery system compound via head and neck. Epidermis and nasal mucosa;

10. The drug delivery system described in accordance with claim 1, 2, 3, 5 or 6 is characterized by, among other things, not limited to cream, medicine drops, hydrophilic gel, gel, film agent, nasal spray, soft paste, and frame paste;

11. The delivery system according to claim 1, wherein the drug delivery system comprises one or more specific nanobodies can be absorbed via skin of the head epidermis and neck and nasal mucosa, enter the blood circulation of the brain and reach the preset lesion site;

12. The drug delivery according to claim 1, wherein the drug delivery system is prepared into the applications that nanobody drug is absorbed via head and neck epidermis and nasal mucosa for the local or adjacent areas;

13. The applications according to claim 11 or 12, including the application that nanobody drug does not need to directly penetrate the BBB, while entering the intracranial brain tissue and central nervous system through the outer cortex of the head and neck epidermis and inner layer of nasal cavity to reach the target lesion and playing the role of a drug delivery;

14. The application according to claim 11 or 12 includes the application that enables nanobody to penetrate head and neck epidermis and penetrate the BBB, entering the blood circulation, and reaching the target lesion site of the CNS to play a treatment role.

15. The application of the drug delivery system in claim 1, wherein the application of the drug delivery system can be prepared for the health maintenance and health care of the brain and nervous system of elderlies.
